(19) United States
(12) Patent Application Publication
(10) Pub. No.: US 2026/0345017 A1
(43) Pub. Date: Nov. 5, 2026

(54) SYSTEM FOR MEASURING BLOOD PRESSURE

(71) Applicant: ROCKLEY PHOTONICS LIMITED, Altrincham (GB)

(72) Inventor: Kate LeeAnn BECHTEL, Pleasant Hill, CA (US)

(21) Appl. No.: 19/426,008

(22) Filed: Dec. 18, 2025

Related U.S. Application Data
(60) Provisional application No. 63/798,419, filed on May 1, 2025.

(51) Int. Cl. A61B 5/022; A61B 5/00; A61B 5/024

(52) U.S. Cl. CPC A61B 5/022; A61B 5/0004; A61B 5/02416; A61B 5/7221; A61B 2560/0223; A61B 2562/0247

(57) ABSTRACT

The utility model discloses a blood flow velocity measuring system capable of synchronously collecting photoelectric laser speckles. The measuring system comprises a laser light source, a first lens, a spectroscope, a laser speckle light path, a photoplethysmography light path, a high-precision analog-digital converter, a camera and a computer. According to the utility model, the photoplethysmography light path is adopted to acquire photoplethysmography signals, the acquisition is not limited by acquisition speed and an analysis method, and the signal-to-noise ratio is extremely high; meanwhile, the laser speckle contrast waveform and the photoplethysmography signal have extremely high similarity and are both generated by cardiac periodic pulsation; therefore, according to the method for reducing the noise by adopting the photoplethysmography signals, the measurement precision of the final blood flow rate can be effectively improved by reducing the noise of the laser speckle contrast waveform through the technologies of filter design, independent component analysis or machine learning and the like.

### CROSS-REFERENCE TO RELATED APPLICATION(S)

The present application claims priority to and the benefit of U.S. Provisional Application No. 63/798,419, filed May 1, 2025, entitled “SYSTEM FOR MEASURING CUFFLESS BLOOD PRESSURE”, the entire content of which is incorporated herein by reference.

### FIELD

One or more aspects of embodiments according to the present disclosure relate to biomarker monitoring, and more particularly to a system for measuring blood pressure.

### BACKGROUND

Blood pressure is a biomarker with clinical significance in various contexts.

It is with respect to this general technical environment that aspects of the present disclosure are related.

### SUMMARY

According to an embodiment of the present disclosure, there is provided a system, including: a cuff including an inflatable bladder and a pressure sensor; and an optical sensor, including a speckleplethysmography (SPG) sensor, the cuff being configured to produce a first time series, of pressure measurements, the optical sensor being configured to produce a second time series, of SPG measurements, and the system being configured to make a determination based on a combination of a representation of the first time series and a representation of the second time series.

In some embodiments, the system includes a band including the optical sensor.

In some embodiments, the system is configured to make the determination based on a combination of the first time series and the second time series.

In some embodiments, the making of the determination includes an operation selected from the group consisting of: determining whether the cuff and the optical sensor are on the same limb, determining whether the first time series and the second time series were produced concurrently, determining whether a signal from the cuff and a signal from the optical sensor are both acceptable, determining whether the cuff was inflated above systolic pressure, and generating subject-specific calibration parameters.

In some embodiments, the cuff is configured to transmit a representation of the first time series or the optical sensor is configured to transmit a representation of the second time series.

In some embodiments, the cuff is configured to transmit the first time series over an encrypted wireless link.

In some embodiments, the optical sensor or the cuff is configured to transmit first data, over a wireless link, to a mobile device, the first data being based on the first time series or the second time series.

In some embodiments, the system further includes the mobile device, wherein the mobile device is configured to transmit second data to a server, the second data being based on the first data.

In some embodiments, the mobile device is configured to prompt a user to complete a registration process.

In some embodiments, the mobile device is configured to prompt a user to confirm that a subject has assumed a correct body position for a blood pressure measurement.

In some embodiments, the optical sensor is further configured to produce a third time series, of PPG measurements.

In some embodiments, the system is configured: to produce the first time series, the second time series, and the third time series concurrently, on a first occasion; to produce, on a third occasion, a seventh time series, of SPG measurements, and an eighth time series, of PPG measurements, without the concurrent production of a time series of pressure measurements; and to estimate a blood pressure, the estimating being based on the first time series, the second time series, the third time series, the seventh time series, and the eighth time series.

In some embodiments: the system is further configured to produce, on a second occasion, a fourth time series, of pressure measurements, a fifth time series, of SPG measurements, and a sixth time series, of PPG measurements; and the estimating is further based on the fourth time series, the fifth time series, and the sixth time series.

In some embodiments, the making of the determination includes determining whether a signal from the cuff and a signal from the optical sensor are both acceptable, and wherein the determining that the signal from the cuff is acceptable includes determining that the cuff is correctly placed on a limb of a subject.

In some embodiments, the cuff is configured to perform, in response to an electronic command received from an external component: cuff inflation; cuff deflation; and during the cuff inflation and the cuff deflation, the producing of the first time series.

In some embodiments, the external component is a mobile device.

In some embodiments, the external component is the optical sensor.

According to an embodiment of the present disclosure, there is provided a system, including: a cuff configured to be secured around a limb of a subject, the cuff including: an inflatable bladder; an air pump for inflating the inflatable bladder; a pressure sensor, for sensing a pressure in the inflatable bladder; and a processing circuit, the cuff being configured: to receive an electronic command, and in response to receiving the electronic command, to perform cuff inflation.

In some embodiments, the cuff is further configured, in response to receiving the electronic command: to perform cuff deflation, and during a portion of the cuff inflation and a portion of the cuff deflation, to obtain a time series of pressure measurements.

In some embodiments, the cuff is further configured to transmit the time series of pressure measurements.

In some embodiments, the cuff is configured to transmit the time series of pressure measurements over an encrypted wireless link.

### BRIEF DESCRIPTION OF THE DRAWINGS

These and other features and advantages of the present disclosure will be appreciated and understood with reference to the specification, claims, and appended drawings wherein:

FIG. 1A is a system-level diagram, according to an embodiment of the present disclosure;

FIG. 1B is a block diagram of an optical sensor, according to an embodiment of the present disclosure;

FIG. 1C is a block diagram of a cuff, according to an embodiment of the present disclosure;

FIG. 2A is a illustration of a wrist and hand showing a region on which an optical sensor may be placed, according to an embodiment of the present disclosure;

FIG. 2B is a illustration of a wrist and hand with an optical sensor, according to an embodiment of the present disclosure;

FIG. 3A is an illustration of a subject and a system for measuring blood pressure during the performance of an initialization procedure, according to an embodiment of the present disclosure; and

FIG. 3B is an illustration of a subject and a system for measuring blood pressure during the performance of a cuffless blood pressure measurement, according to an embodiment of the present disclosure.

### DETAILED DESCRIPTION

The detailed description set forth below in connection with the appended drawings is intended as a description of exemplary embodiments of a system for measuring blood pressure provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

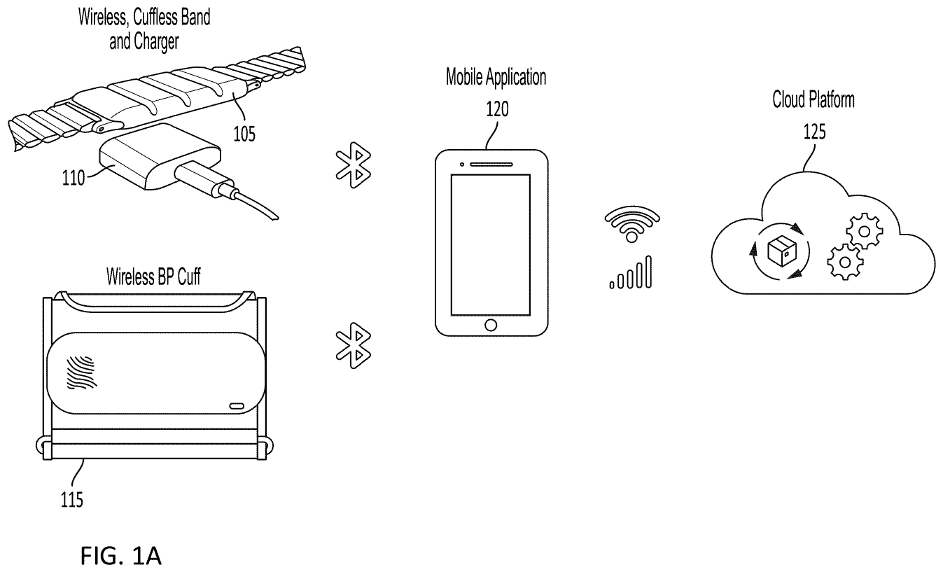

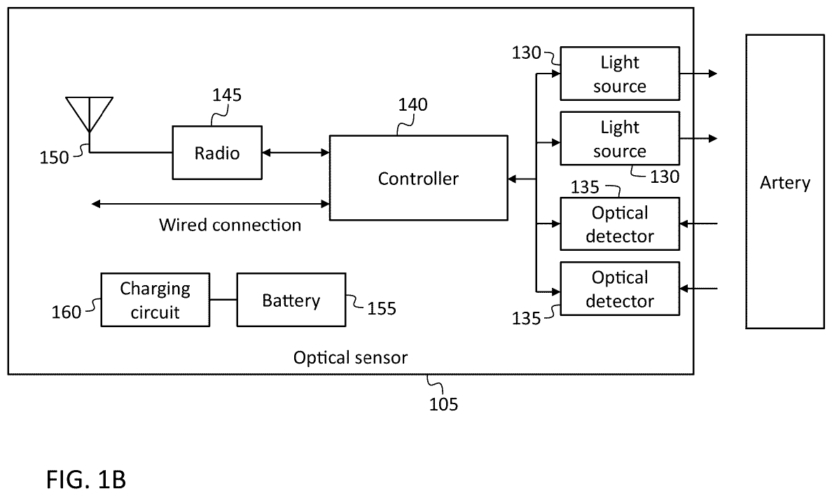

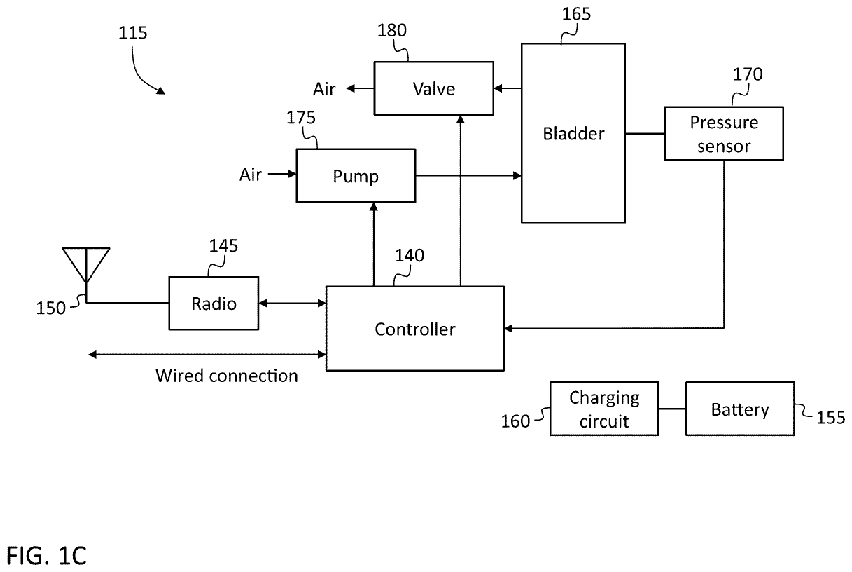

FIG. 1A is a system-level diagram of a system for measuring blood pressure in some embodiments. The system may include a wireless, battery-operated, band (or “cuffless band”) including an optical sensor (or “optical sensor module”) 105 and its associated charger 110, a wireless, battery-operated automated cuff (or “inflatable cuff”) 115, a mobile device 120 (e.g. a mobile telephone or other mobile computing device such as a tablet computer or laptop computer) running a mobile application, and a cloud platform 125 (e.g., one or more servers connected to the internet, and associated software, running on the servers). The band may include a strap and the optical sensor 105. The strap may be secured around the wrist, so that the optical sensor 105 is over the radial artery at the wrist. As used herein, a “sensor” is a device for sensing; as such a “sensor”, as the term is used herein, may include one or more sensors. For example, the optical sensor 105 may include one or more speckleplethysmography (SPG) sensors or one or more photoplethysmography (PPG) sensors for performing optical measurements on the radial artery. The cuff 115 may contain an inflatable bladder for applying pressure to a limb (e.g., an arm) of the subject and a pressure sensor. The cuff 115 may be worn over the upper arm and may be utilized for periodic initialization (or “calibration”) measurements. The mobile application may communicate with the optical sensor 105 and with the cuff 115 via a wireless Bluetooth low energy (BLE) connection. The mobile application may transfer data to and receive data from the cloud platform over a cellular or Wi-Fi connection. In some embodiments, either or both of the optical sensor 105 and the cuff 115 is worn elsewhere on the arm or on another limb; for example, the cuff 115 may be worn on the forearm or on a leg, or the optical sensor 105 may be worn over the ulnar artery, or both the cuff 115 and the optical sensor 105 may be worn on a leg.

Referring to FIG. 1B, the optical sensor 105 may include (i) one or more light sources 130 for illuminating one or more blood vessels in a patient, or “subject”, and (ii) one or more optical detectors 135 (each of which may be or include, for example, an image sensor (e.g., a CMOS image sensor (CIS)), or a photodiode (PD) for receiving light scattered from the blood vessel (e.g., from blood inside an artery). The health sensor may include (i) a speckleplethysmography (SPG) sensor including a first light source 130 (which may be or include a source of coherent light, e.g., a laser) and a first optical detector 135 (which may be or include a complementary metal oxide semiconductor (CMOS) image sensor (CIS) or a charge-coupled device (CCD)), for measuring the instantaneous blood velocity in the blood vessel, and (ii) a photoplethysmography (PPG) sensor including a second light source 130 (which may be or include a source of incoherent light, e.g., a light-emitting diode (LED)) and a second optical detector 135 (which may be or include a single-pixel photodetector such as a photodiode (PD)), for measuring the instantaneous blood volume in a portion of the blood vessel. Each of the instantaneous blood velocity in the blood vessel, and the instantaneous blood volume may vary with the cardiac cycle. A blood pressure (e.g., the systolic blood pressure or the diastolic blood pressure, or both) may be inferred from a time history (or “time series”) of instantaneous blood velocity measurements and a time history of instantaneous blood volume measurements. In some embodiments, a single (shared) light source is used for both the SPG sensor and the PPG sensor (e.g., a light source that is configured to selectively produce either coherent light or incoherent light, or a light source that is configured to produce coherent light (which may be used for both the SPG and PPG measurements). In some embodiments, a single (shared) optical detector 135 is used for both the SPG sensor and the PPG sensor. Such a sensor may be or include, for example, an image sensor with a high dynamic range analog to digital converter or an image sensor with a circuit for performing DC subtraction prior to analog to digital conversion.

In some embodiments, the optical sensor 105 includes two sensors with different orientations. For example, the optical sensor 105 may include (i) a first sensor (e.g., an SPG sensor) including a first light source 130 and a first optical detector 135, the first light source 130 and the first optical detector 135 being separated along a direction substantially parallel (e.g., within 30 degrees of being parallel) to the blood vessel, and (ii) a second sensor (e.g., a PPG sensor) including a second light source 130 and a second optical detector 135, the second light source 130 and the second optical detector 135 being separated along a direction substantially perpendicular (e.g., within 30 degrees of being perpendicular) to the blood vessel. In such a configuration the second sensor may be positioned along the blood vessel such that a centroid of the blood volume with which the first sensor interacts is at substantially the same point along the length of the blood vessel as a centroid of the blood volume with which the second sensor interacts (e.g., the centroids may be separated by less than three times the diameter of the blood vessels).

The optical sensor 105 may further include a controller 140, which may be or include a processing circuit, and which may control the light sources 130 and receive data from the optical detectors 135, and communicate with other components of the system (e.g., with the cuff 115, with the mobile device 120, or with the cloud platform 125), through (i) a radio 145 connected to (or including) an antenna 150 or (ii) a wired connection (e.g., a temporary wired connection established by connecting a cable between the mobile device 120 and the optical sensor 105). The elements of the optical sensor 105 may be powered by a battery 155 (e.g., a rechargeable battery, which may periodically be recharged through a charging circuit which may include, or be connected to, a coil for wireless battery charging. During charging, this coil may operate as the secondary winding of a charging transformer the primary winding of which is part of the charger 110.

The controller 140 may periodically (e.g., during intervals when the second light source 130 is turned off) perform one or more SPG measurements, by turning on the first light source 130 and reading data from the first optical detector 135, and it may periodically (e.g., during intervals when the first light source 130 is turned off) perform one or more PPG measurements, by turning on the second light source 130 and reading data from the second optical detector 135. In some embodiments, the first light source is not turned off (e.g., it remains on) when a PPG measurement is preformed, or the second light source is not turned off (e.g., it remains on) when an SPG measurement is performed. SPG and PPG measurements may be performed alternately, e.g., during initialization, or during a cuffless blood pressure measurement, yielding, during such a time interval, a time series of SPG measurements and a time series of PPG measurements. In some embodiments, the first optical detector 135 is an image sensor that, for each measurement, performs an exposure during a first time interval (which may be referred to as an “exposure interval”), and then performs a readout operation during a second time interval. The first and second time intervals may overlap (e.g., if the image sensor is a rolling-shutter image sensor). In some embodiments, PPG measurements are made (e.g., the second light source is turned on, and data is read from the second optical detector 135 (and the first light source may be turned off)) during the intervals between consecutive exposure intervals.

In other embodiments, the SPG and PPG measurements may be performed in a different sequence. For example, the SPG and PPG measurements may overlap (with the first light source 130 being used for both SPG and PPG measurements), or some PPG measurements may be performed using the first light source 130 (and each of these PPG measurements may or may not overlap with an SPG measurement) and some PPG measurements may be performed using the second light source 130. In some embodiments the SPG and PPG measurements are performed at different respective rates. In such an embodiment (i) some or all of the SPG and PPG measurements may overlap, or (ii) some or all of the measurements may be non-overlapping. Each of (i) the rate at which PPG measurements are made and (ii) the rate at which SPG measurements are made may be between 20 Hz and 1000 Hz (e.g., between 20 measurements per second and 1000 measurements per second). In some embodiments, a plurality of measurements is performed each time a light source 130 is turned on. For example, during a first time interval, the first light source 130 may be turned on, and a plurality of images may be read from the first optical detector 135. These images may then be used to calculate a first SPG measurement (e.g., from an average speckle contrast calculated from the images). During a second time interval, the second light source 130 may be turned on, and a plurality of photocurrent values may be read from the second optical detector 135. These photocurrent values may then be used to calculate a first PPG measurement (which may be an average PPG measurement for the second time interval).

The controller 140 may also receive instructions from one or more of the other components of the system, or it may send instructions to one or more of the other components of the system. For example, in an embodiment in which the mobile device 120 manages the operation of the system, the controller 140 of the optical sensor 105 may receive a command to perform a series of SPG measurements and a series of PPG measurements, and to send the resulting data to the mobile device (which may also control the cuff 115 as needed). In an embodiment in which the optical sensor 105 manages the operation of the system, the controller 140 of the optical sensor 105 may perform SPG and PPG measurements (using the light sources 130 and the optical detectors 135), and, during an initialization process, it may instruct the cuff 115 (i) to inflate (e.g., to pump air into the cuff bladder so as to increase the pressure in the bladder) or (ii) to deflate (e.g., to allow air out of the cuff bladder so as to decrease the pressure in the bladder), (iii) to obtain a time series of pressure measurements during inflation or during deflation, and (iv) to send the resulting data to the processor in the optical sensor 105, or to another component of the system (e.g., to the mobile device 120, which may relay the data to the cloud platform 125).

Referring to FIG. 1C, the cuff 115 may include (as mentioned above) an inflatable bladder 165 and a pressure sensor 170 for sensing (e.g., measuring) the pressure in the bladder. Like the optical sensor 105, the cuff 115 may include a controller 140, which, in the cuff 115, may control (i) the pump 175 to pump air into the inflatable bladder 165 or (ii) the valve 180 to allow air to escape from the bladder 165. The pressure sensor 170 may be capable of performing (e.g., may have a bandwidth sufficient to perform) pressure measurements at a sample rate of at least 1 Hz (e.g., at a sample rate of at least 10 Hz, or at a sample rate of between 1 Hz and 10 kHz).

The elements of the cuff 115 (like those of the optical sensor 105) may be powered by a battery 155 (e.g., a rechargeable battery, which may periodically be recharged through a charging circuit. The controller 140 of the cuff 115 may receive instructions from one or more of the other components of the system, or it may send instructions to one or more of the other components of the system. For example, in an embodiment in which the mobile device 120 manages the operation of the system, the controller 140 of the cuff 115 may receive a command to perform a series of pressure measurements while inflating the inflatable bladder 165 or deflating the inflatable bladder 165, and to send the resulting data to the mobile device (which may also control the optical sensor 105 as needed). In an embodiment in which the cuff 115 manages the operation of the system, the controller 140 of the cuff 115 may, during initialization, perform pressure measurements while inflating and deflating the inflatable bladder 165, and it may instruct the optical sensor 105 (i) to perform, concurrently with the inflating and deflating, a plurality of PPG and SPG measurements, and (ii) to send the resulting data to the cuff 115, or to another component of the system (e.g., to the mobile device 120).

In some embodiments, the cuff 115 is self-contained, in the sense that all of the components of the cuff 115 which are illustrated in FIG. 1C are integral with (e.g., sewn into or attached directly to) a fabric holder (e.g., none of the components is secured to the fabric holder only by a cable or hose). The fabric holder may contain the inflatable bladder 165, and include a fastener (e.g., one or more strips of hook and loop fasteners, or a strap and a buckle) for securing the cuff 115 to the limb (e.g., to the arm) of the subject.

The cloud platform 125 may provide data storage and analysis services. For example, the cloud platform 125 may receive (i) on a first occasion (during initialization) a first time series, of cuff pressure measurements (e.g., during cuff inflation and cuff deflation), from the cuff 115, a second time series, of SPG measurements, from the optical sensor 105, and a third time series, of PPG measurements, from the optical sensor 105, and (ii) on a second occasion (e.g., during a second initialization measurement), a fourth time series, of cuff pressure measurements during cuff inflation and deflation, a fifth time series, of SPG measurements, from the optical sensor 105 and a sixth series, of PPG measurements, from the optical sensor 105. The cloud platform 125 may use this information to generate the initialization (calibration) parameters for the subject. The second initialization process may be a repetition of the initialization process, which may be performed to obtain more accurate data (e.g., by averaging data from the first initialization process and the second initialization process), or to assess the reliability of the initialization data (e.g., by assessing the repeatability of the initialization data).

As used herein, a calculation is “based on a combination of” a first set of data and a second set of data if the first set of data (e.g., a first time series) and the second set of data (e.g., a second time series), or data (such as encrypted versions or compressed versions (using lossless compression) of one or the other or both of the first set of data and the second set of data) from which these sets of data may be inferred, may be concurrently present within (or available to) a processing circuit (e.g., within one of the four active components of the system of FIG. 1A (e.g., one of the optical sensor 105, the cuff 115, the mobile device 120, and the cloud platform 125)). The initialization parameters may be generated based on a combination of the first time series and the second time series (e.g., based on a combination of the first time series, the second time series, and the third time series) in the sense that the first time series and the second time series, or data (such as encrypted versions of one or the other or both of the first time series and the second time series) from which these time series may be inferred, may be concurrently present within (or available to) one of the four active components of the system of FIG. 1A (e.g., one of the optical sensor 105, the cuff 115, the mobile device 120, and the cloud platform 125). As such, an embodiment involving generating the initialization parameters based on a combination of the first time series and the second time series may be distinct from, e.g., an embodiment in which a blood pressure is first calculated, based on the first time series, and the calculated blood pressure (which may be insufficient to infer the first time series) is then combined, in the cloud platform 125, with the second time series.

As used herein, a “representation” of a first set of data (e.g., of a first time series) is a second set of data, from which it is possible to reconstruct each of at least N points of the first set of data with an accuracy of X % or better, where N is between 2 and 10,000 and X is between 0.01 and 50 (corresponding to 0.01% and 50%, respectively). For example, N may be 5 and X % may be 10%. As such, a representation of a time series may be (i) the time series itself, or (ii) a compressed version of the time series (e.g., a compressed version generated using loss compression) or (iii) coefficients of a polynomial spline generated by fitting to the time series.

After initialization, upon further use of the optical sensor 105 alone, the blood pressure may be estimated (e.g., calculated) from the newly obtained measurements, e.g., from (i) a seventh time series, of SPG measurements, from the optical sensor 105, (ii) an eighth time series, of PPG measurements, from the optical sensor 105, and (iii) the previously generated initialization data. Blood pressure may be estimated from SPG and PPG timeseries data in various ways, for example as described in: Ariane Garrett, Byungchan Kim, Edbert J. Sie, Nil Z. Gurel, Francesco Marsili, David A. Boas, and Darren Roblyer, “Simultaneous photoplethysmography and blood flow measurements towards the estimation of blood pressure using speckle contrast optical spectroscopy,” Biomed. Opt. Express 14, 1594-1607 (2023). Additional biometrics (beyond blood pressure), such as arterial stiffness, may be calculated from the same or similar data, and biometrics such as the presence or severity of cardiac dysrhythmias or aortic stenosis may be obtained from the same or similar data.

After initialization, the system may measure systolic and diastolic blood pressure and pulse rate on demand using measurements performed by the optical sensor 105. For example, optical measurements (e.g., PPG and SPG measurements) of the radial artery, performed by the optical sensor 105, may be used to calculate blood pressure and pulse rate. As used herein, the “subject” is the person whose blood pressure is measured; the “user” is the person operating the system. As such, the user may be the subject, or, for example, a clinician.

Before performing the first measurement, the user may complete a registration process in the mobile application to securely define a user profile. After registration, the optical sensor 105 and cuff 115 may be connected (e.g., to each other, or to the mobile device, e.g., via BLE) and a unique device identifier of each of the optical sensor 105 and cuff 115 may be tied to the user's account. Various combinations of connections between the four active components of FIG. 1A (the optical sensor 105, the cuff 115, the mobile device 120, and the cloud platform 125) may be established and used. In some embodiments, each of the active components is connected (directly or indirectly) to all three of the other active components. In some embodiments, there is more than one communication path between two of the active components; for example, the mobile device 120 may be connected (directly, through BLE) to the optical sensor 105 and to the cuff 115, (providing an indirect connection between the optical sensor 105 and to the cuff 115), and the optical sensor 105 may also be directly connected to the cuff 115 (e.g., by a BLE connection). Once the registration process is complete, the communication links between the active components may be sufficiently secure to safeguard the health-related information that may be transmitted through these links. Any data that is stored in any of the active components may also be encrypted.

The initialization procedure may be performed periodically, for example once every 21, 28, or 60 days. As mentioned above, during initialization, information (e.g., measurements) from the cuff 115 and from the optical sensor 105 may be obtained concurrently (e.g., the time interval during which the cuff measurements are obtained may overlap with the time interval during which the optical sensor measurements are obtained). These measurements, or data derived from these measurements, may be referred to as “initialization data”. The cuff and optical sensor measurements obtained during initialization may be stored for use in calculating the blood pressure on other occasions (e.g., future or past occasions) when measurements are performed using only the optical sensor 105.

During initialization, the cuff may be inflated and deflated, and cuff pressure measurements and SPG and PPG measurements may be recorded. Each of these measurements may be performed and recorded repeatedly, to form a time series (e.g., with a sample rate of between 4 Hz and 1 kHz), which may be stored, or derivative data which may be stored. Derivative data may be any data derived from any of the time series or from any combination of the time series. In some embodiments, the initialization process is repeated multiple times in one sitting, generating several corresponding sets of initialization data. The number of repetitions may be specified by the user. The system may ensure that at least 30 seconds has passed after a cuff deflation before any successive cuff inflation, to avoid venous congestion. The system may require that multiple cuff-determined blood pressure values (e.g., the first and second cuff-determined blood pressure values if the initialization process is performed twice) be within 12 mmHg systolic and 8 mmHg diastolic.

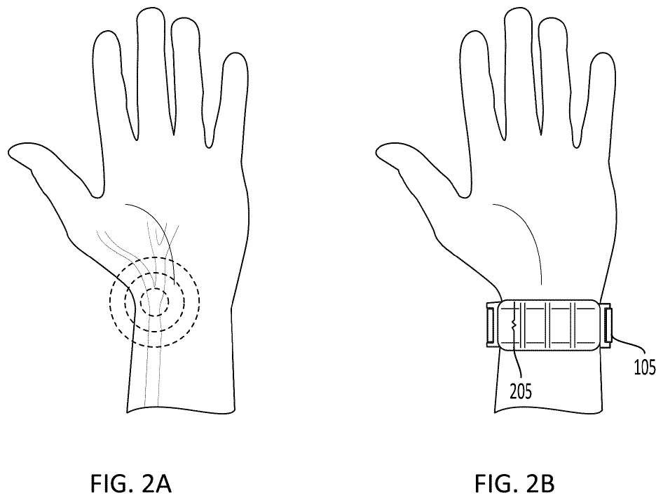

The mobile application may guide the user through the initialization process, which may begin with the user placing the cuff 115 on the upper arm and the optical sensor 105 on the wrist of the same arm. Visualization and instruction within the mobile application may guide the user to place the optical sensor 105 over the radial artery. Either palpation or anatomy may be used to guide the placement. The optical sensor 105 may have an alignment marker 205 on the top of the optical sensor 105 to aid in the placement. FIG. 2A is an illustration of the location of the radial artery, and FIG. 2B is an illustration of the optical sensor 105 positioned such that alignment marker 205 on the optical sensor 105 is placed over the radial artery.

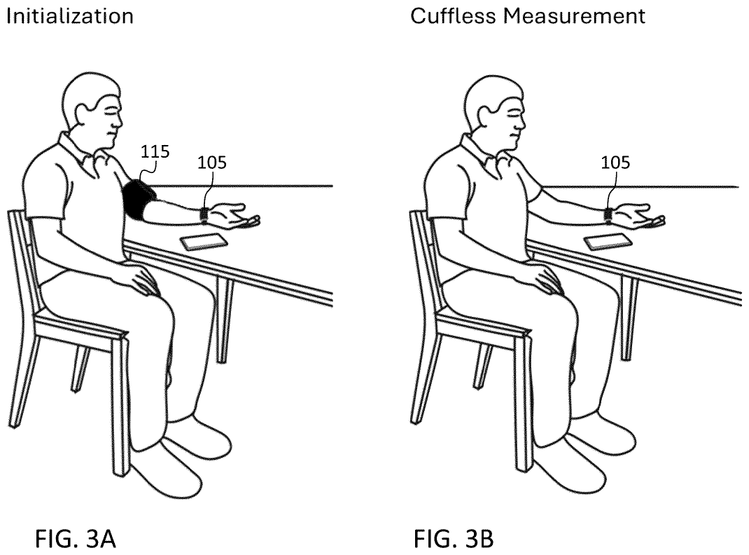

During initialization, and during optical sensor-only measurements, the mobile application may illustrate to the subject the proper body position via American Heart Association (AHA) guidelines. For example, the defined body position may be seated at rest, legs uncrossed with feet flat on the floor, with the arm supported and palm facing upward on a flat surface such as a table (or on the arm of a chair in which the subject is seated, or on the subject's lap), as shown in FIG. 3A (for initialization) and FIG. 3B (for an optical sensor-only (or “cuffless”) blood pressure measurement). The wrist bearing the optical sensor 105 does not need to be at heart level, but rather at any reproducible height with respect to the chest, which is the same as when the initialization data was acquired. In some embodiments, the optical sensor data is referenced to the cuff 115 and is not a direct measure of blood pressure, and, as such, the hydrostatic difference in pressure (e.g., between the wrist and the upper arm) is inherently accounted for.

Before an initialization measurement and before an optical sensor-only measurement, the subject may be instructed to rest quietly for 5 minutes per AHA guidelines for accurate blood pressure measurements. The subject may have the option to skip this step as the subject may be already rested. During the rest time, if not skipped, the mobile application may provide educational content to the subject to allow the subject to learn more about hypertension and health management.

Before an initialization measurement and before an optical sensor-only measurement, data may be acquired from the optical sensor 105 to ensure adequate pulsatile flow detection. If the optical signal quality is not adequate as determined by a calculated signal quality metric (e.g., a metric that takes into account pulse amplitude, frequency content, motion, or pulse morphology), the user may be instructed to reposition the device, e.g., the mobile application may provide feedback to the user for optimal placement of the optical sensor 105 over the radial artery. During initialization, after adequate optical signal quality is obtained, the mobile application may command the cuff 115 (e.g., through a wireless link, such as a BLE link) to begin an automated cycle of inflation and deflation and calculation of systolic and diastolic blood pressure and pulse rate. In some embodiments the cuff transmits (e.g., to the mobile device 120) not only the measured systolic and diastolic blood pressure and pulse rate, but also the full time series of pressure sensor (or “pressure transducer”) data from inflation through deflation. The cuff pressure transducer time series data may be used in conjunction with the optical time series data acquired from the optical sensor 105 at the same time for one or more of, e.g., at least the following purposes: (i) confirming that the initialization data is taken by the subject, (ii) preventing erroneous cuff readings, and (iii) obtaining individualized calibration factors (based on the pressure time series, the SPG time series, or the PPG time series).

Confirming that the initialization data is taken by the subject may be advantageous, e.g., because a subject may not wish to acquire a cuff reading as part of the initialization process due to discomfort or disinterest. As such, in a system in which the user is only required to enter (e.g., type) a blood pressure reading into the device (or mobile application), there is a risk that the user may generate false numbers, which may lead to inaccuracy of the generated calibration factors and, therefore, of subsequent measurement results. Similarly, in a system in which the blood pressure is automatically and wirelessly read from the cuff (e.g., to mitigate the user-entry error), a risk may remain that the subject did not wear the cuff or wore it incorrectly. For example, the subject may have given the cuff to another person to take the reading or may have worn the cuff on an opposite limb. An opposite arm may have a lateral difference in blood pressure to the same arm and the optical data may have different characteristic morphology. As such, these situations may similarly result in inaccuracies in the generated calibration factors and therefore in subsequent measurement results.

Some embodiments eliminate or reduce such risks by analyzing the cuff pressure time series and the optical sensor signals together. For example, the presence of heartbeats in the optical data may be seen to diminish with the increase in cuff pressure. The optical data baseline values may also be seen to change according to applied cuff pressure. For example, blood flow velocity may reduce as cuff pressure is increased. Upon cuff deflation, the heartbeats may be observed to reappear and the baseline flow may be observed to increase, both eventually returning to near their respective original values, with or without a hyperemia response. These characteristic signals, if observed, may demonstrate that the blood pressure results obtained from the cuff are indeed obtained from the same limb (e.g., the same arm) as the optical sensor 105 and at the same time, thereby reducing error in the generated calibration factors.

As mentioned above, combining the cuff pressure transducer time series data with the optical time series data acquired from the optical sensor 105 at the same time may prevent erroneous cuff readings. For example, some subjects may not obtain accurate cuff (oscillometric) blood pressure results due to improper cuff sizing or placement; in such a situation, comparing the cuff pressure time series to the optical sensor signals may allow the system to determine that the cuff measurement is not reliable. For example, the downstream (wrist position) optical signals, and in particular the blood velocity signal, generates sharp waveforms with each heartbeat. The magnitudes of these signals are seen to decrease with increasing cuff pressure until, once cuff pressure is higher than systolic pressure, the heartbeat signals are no longer observed in the optical data. Checking for this characteristic in the optical signals may make it possible to avoid using data obtained when the cuff did not in fact inflate to a pressure above the systolic pressure. Further, the optical signals, and again in particular the blood velocity signal, may be seen to result in observable heartbeats as soon as the cuff pressure deflates to or below systolic pressure.

When any indications of improper cuff placement are detected, the user may be informed that the initialization data was invalid and needs to be re-acquired. The user may then be prompted to confirm cuff position, cuff size and fit, optical sensor position, that the cuff 115 is on the same arm as the optical sensor 105, and body position, and to re-acquire the initialization data.

As mentioned above, combining the cuff pressure transducer time series data with the optical time series data acquired from the optical sensor at the same time may make it possible to obtain additional individualized calibration factors. The behavior of the optical sensor signals during increasing and decreasing external pressure applied by the cuff provides additional information that may be used to improve algorithm accuracy for a subject. For example, the cuff pressure time series combined with SPG, PPG, or other optical signals from the optical sensor 105 may be used to extract pressure mediated flow or volume information (e.g., may be used to measure changes in flow or volume with changes in cuff pressure) for personalized calibration factors to improve algorithm results.

The cuff pressure time series and the optical measurement time series generated by the optical sensor 105 may be capable of being aligned in time to generally within one heartbeat (e.g., to an accuracy that is better than (i.e., that results in a synchronization error less than) a value between 1 second and 10 ns). The exact timing requirement may depend on the user's pulse rate and the rate of cuff inflation and deflation. In some embodiments, in which BLE is used for communication between the optical sensor 105, the mobile device 120, and the cuff 115, a different signal (e.g., a cellular signal, a Wi-Fi signal, or a GPS signal) may be used for synchronization.

In some embodiments, the cuff 115 and the optical sensor 105 are both controlled such that data is acquired from each with the appropriate timing. For example, the optical sensor 105 may be instructed to begin collecting data prior to cuff inflation so that the change in signal with inflation is evident in the optical data. In some embodiments, between 20 seconds and 40 seconds worth of data may be obtained prior to cuff inflation. The cuff 115 may then be instructed wirelessly to begin inflation. This instruction may be an electronic command sent via the mobile application (or directly from the optical sensor 105) rather than a manual command given by (e.g. a button press performed by) the subject. Such a method for triggering cuff inflation may be advantageous because if an action on the part of the subject (e.g., pushing a button on the cuff) were needed to trigger cuff inflation, then the movement of the subject to perform this action could compromise the optical data being acquired. Automated electronic (e.g., wireless) triggering of the cuff may also be more convenient for the subject, and more reliable.

In some embodiments, the cuff is configured to perform inflation or deflation such that the pressure, during deflation, is a linear function (or a known non-linear function) of time. In such an embodiment it may be possible for the system to infer the cuff pressure, at any given time, with greater accuracy.

After initialization data has been acquired and saved in the cloud platform 125, cuffless blood pressure measurements may be acquired on demand by the user. Cuffless (e.g., optical sensor-only) blood pressure measurements may, as mentioned above, be performed using a method similar to the method used for initialization measurements, except that the cuff may not be used. The system may prevent a cuffless (optical sensor-only) measurement from proceeding if the initialization data is older than a pre-set duration (e.g. a duration between 1 day and 180 days). The mobile application may request user confirmation of correct body position and request that the user agree to stay still and avoid talking during data acquisition. Following user confirmation of optical sensor and body position in the mobile application, the optical sensor 105 may acquire optical data.

The mobile application may present questions or selection options of common symptoms, events, and health status, along with the option for free text user notes, after a reading (e.g., a set of time series of optical data (e.g., an SPG time series and a PPG time series)) is acquired. This way, the user may record any symptoms or dietary habits that may have influenced the reading. User notes entry may occur before the results for a reading are displayed so as not to influence the user to inaccurately justify the result.

After a reading is obtained, the optical data may be sent to the cloud platform 125, and measurement results (e.g., blood pressure or pulse rate) may be displayed in the mobile application within 30 seconds after conclusion of data acquisition, if cloud connectivity exists. In the absence of cloud connectivity, the data may be stored locally and transmitted once connectivity is re-established. The user may be notified of the lack of connectivity and that results may become available once connectivity is re-established. In some embodiments, cloud connectivity is required for a measurement to begin. In other embodiments, algorithms are performed locally on the optical sensor 105 or by the mobile application and cloud connectivity is not required for viewing results.

If a result is invalid, which may occur if there is insufficient data quality to generate a result or if the result is out of range of the systolic or diastolic pressure ranges supported by the device, the user may be informed of the issue, and given an explanation of one or more possible causes, as well as remedial actions the user may attempt.

The mobile application may both display the current result and allow the user to view historical trends. Additionally, the mobile application may allow the user to generate a portable document format (pdf) report with selectable date ranges that the user may choose to share with a doctor. Such a report may follow European Society of Hypertension (ESH) and AHA guidelines.

Synchronization and control of the optical sensor 105, the mobile device 120, and the cuff 115 may be performed by any one of these three devices. For example, when an initialization is to be performed, the mobile device 120 may instruct the optical sensor 105 to begin performing optical measurements and to store or transmit the corresponding time series, and, after some time has elapsed, the mobile device 120 may instruct the cuff 115 to begin collecting a pressure time series and to begin an inflation and deflation sequence. In other embodiments, the optical sensor 105 may control this sequence and send instructions to the cuff 115 accordingly. Similarly, the processing of the time series obtained by the optical sensor 105 and the cuff 115 may be performed within any one of the four active components of the system of FIG. 1A (e.g., one of the optical sensor 105, the cuff 115, the mobile device 120, and the cloud platform 125). In any such embodiment any time series that is not generated by the device performing the processing may be transmitted (or a representation of the time series may be transmitted) to the device performing the processing. For example, if the mobile device 120 is configured to generate blood pressure estimates from the time series generated by the optical sensor 105 and by the cuff 115, then each of the optical sensor 105 and the cuff 115 may transmit the time series it generates to the mobile device 120. In some embodiments the mobile device 120 is not used, and the optical sensor 105 or the cuff 115 communicates directly with the cloud platform 125 (e.g., via a Wi-Fi or cellular connection), or the cloud platform 125 is not used (and the analysis and storage of data may be performed by the mobile device 120, for example). In some embodiments, neither a mobile device 120 nor a cloud platform 125 is used, and the storage and analysis of data is performed by the optical sensor 105 or by the cuff 115.

In some embodiments, the mobile device 120 or the optical sensor 105 may provide medication tracking within the application, and the mobile device 120 or the optical sensor 105 may schedule reminders to help the user remember to take blood pressure measurements. The mobile application may allow the user to select a date range within which to display results, e.g., for a health care provider interested in seeing results since a last visit, or since a medication change. The mobile application may allow the user to set (e.g., program) time ranges within a day for aggregating data; for example morning data may be aggregated separately from evening data. The mobile application may conglomerate user notes into actionable feedback as it relates to blood pressure and lifestyle habits.

Some embodiments provide blood pressure measurements that are superior in various ways over other, e.g., related art, methods. For example, each combination of the features identified in the Summary of the present document contributes to providing blood pressure measurements that are more convenient and result in less discomfort, than a sphygmomanometer measurement. As such, each such combination of features results in an improvement in the technology of blood pressure measurement.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X−Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Methods described herein may be performed by one or more processing circuits. Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

It will be further understood that the terms “comprises” or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the inventive concept refers to “one or more embodiments of the present disclosure”. Also, the term “exemplary” is intended to refer to an example or illustration. As used herein, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system for measuring blood pressure have been specifically described and illustrated herein, many modifications and variations will be apparent to those skilled in the art. Accordingly, it is to be understood that a system for measuring blood pressure constructed according to principles of this disclosure may be embodied other than as specifically described herein. The invention is also defined in the following claims, and equivalents thereof.

1. A system, comprising:
a cuff comprising an inflatable bladder and a pressure sensor; and
an optical sensor, comprising a speckleplethysmography (SPG) sensor,
the cuff being configured to produce, on a first occasion, a first time series of cuff pressure measurements, each of the cuff pressure measurements being a measurement of pressure in the inflatable bladder,
the optical sensor being configured to produce, on the first occasion, a second time series of SPG measurements, and
the system being configured to generate subject-specific initialization parameters based on a combination of a representation of the first time series of the first occasion and a representation of the second time series of the first occasion,
wherein the system is configured:
to produce the first time series of the first occasion and the second time series of the first occasion concurrently;
to produce, on a second occasion, a first time series of SPG measurements without concurrently producing a time series of cuff pressure measurements; and
to estimate a blood pressure based on the first time series of the second occasion and the subject-specific initialization parameters.

2. The system of claim 1, comprising a band comprising the optical sensor.

3. The system of claim 1, wherein the system is configured to generate the subject-specific initialization parameters based on a combination of the first time series of the first occasion and the second time series of the first occasion.

4. The system of claim 1, wherein the system is further configured to make a determination based on a combination of the representation of the first time series of the first occasion and the representation of the second time series of the first occasion,
wherein the making the determination comprises an operation selected from the group consisting of: determining whether the cuff and the optical sensor are on the same limb, determining whether the first time series and the second time series were produced concurrently, determining whether a signal from the cuff and a signal from the optical sensor are both acceptable, and determining whether the cuff was inflated above systolic pressure.

5. The system of claim 1,
wherein the cuff is configured to wirelessly transmit the representation of the first time series to the optical sensor or to a mobile device, or the optical sensor is configured to wirelessly transmit the representation of the second time series to the cuff or to a mobile device.

6. The system of claim 5, wherein the cuff is configured to transmit the first time series over an encrypted wireless link.

7. The system of claim 1, wherein the optical sensor or the cuff is configured to transmit first data, over a wireless link, to a mobile device, the first data being based on the first time series of the first occasion or the second time series of the first occasion.

8. The system of claim 7, further comprising the mobile device, wherein the mobile device is configured to transmit second data to a server, the second data being based on the first data.

9. The system of claim 8, wherein the mobile device is configured to prompt a user to complete a registration process.

10. The system of claim 8, wherein the mobile device is configured to prompt a user to confirm that a subject has assumed a correct body position for a blood pressure measurement.

11. The system of claim 1, wherein the optical sensor is further configured to produce, on the first occasion, a third time series of PPG measurements.

12. The system of claim 11, wherein the system is configured:
to produce the first time series of the first occasion, the second time series of the first occasion, and the third time series of the first occasion concurrently;
to produce, on the second occasion, a second time series of PPG measurements; and
to estimate the blood pressure further based on the third time series of the first occasion and the second time series of the second occasion.

13. The system of claim 12, wherein:
the system is further configured to produce, on a third occasion, a first time series, of cuff pressure measurements, a second time series, of SPG measurements, and a third time series, of PPG measurements; and
the generating the subject-specific initialization parameters is further based on the first time series of the third occasion, the second time series of the third occasion, and the third time series of the third occasion.

14. The system of claim 4, wherein the making of the determination comprises determining whether a signal from the cuff and a signal from the optical sensor are both acceptable, and wherein the determining that the signal from the cuff is acceptable comprises determining that the cuff is correctly placed on a limb of a subject.

15. The system of claim 1,
wherein the cuff is configured to perform, in response to an electronic command wirelessly received from an external component:
cuff inflation;
cuff deflation; and
during the cuff inflation and the cuff deflation, the producing of the first time series.

16. The system of claim 15, wherein the external component is a mobile device.

17. The system of claim 15, wherein the external component is the optical sensor.

18-21. (canceled)

22. The system of claim 5, wherein the cuff is configured to wirelessly transmit the representation of the first time series to the mobile device, and the optical sensor is configured to wirelessly transmit the representation of the second time series to the mobile device.

23. The system of claim 5, wherein the cuff is configured to wirelessly transmit the representation of the first time series to the optical sensor.

24. The system of claim 5, comprising a processing circuit,
wherein the system is configured for the processing circuit to:
access both the representation of the first time series and the representation of the second time series; and
generate subject-specific initialization parameters based on the combination of the representation of the first time series and the representation of the second time series.

25. The system of claim 1, wherein the system is further configured to:
produce, on a third occasion, a first time series, of cuff pressure measurements, and a second time series of SPG measurements; and
assess, based on the first and second time series of the first occasion and the first and second time series of the third occasion, the reliability of the first and second time series of the first occasion.
